(19) United States
(12) Patent Application Publication
(10) Pub. No.: US 2026/0248464 A1
(43) Pub. Date: Aug. 27, 2026

(54) DISPLAY APPARATUS AND METHOD FOR MEASURING BIOMETRIC INFORMATION THEREOF

(71) Applicant: Samsung Electronics Co., Ltd., Suwon-si (KR)

(72) Inventors: Yunjoo CHOI, Suwon-si (KR); Donghoon LEE, Suwon-si (KR); Dongwon KIM, Suwon-si (KR); Sangshin PARK, Suwon-si (KR); Eunae CHO, Suwon-si (KR)

(73) Assignee: Samsung Electronics Co., Ltd., Suwon-si (KR)

(21) Appl. No.: 19/648,806

(22) Filed: Apr. 15, 2026

Related U.S. Application Data
(63) Continuation of application No. PCT/KR2024/016772, filed on Oct. 30, 2024.

(30) Foreign Application Priority Data
Nov. 15, 2023 (KR) 10-2023-0158419

(51) Int. Cl. A61B 5/00; A61B 5/0205; A61B 5/11

(52) U.S. Cl. CPC A61B 5/7435; A61B 5/0015; A61B 5/0077; A61B 5/0205; A61B 5/1128; A61B 5/7267

(57) ABSTRACT

A display apparatus including a processor configured to control the display to display content according to a user input, obtain data associated with a usage state of a user based on image data captured by a camera and sensing values of at least one sensor, and based on the usage state being identified as suitable to measure biometric information based on data associated with the usage state of the user and an artificial intelligence model, newly obtain image data of the user through a camera, and obtain the biometric information based on the newly obtained image data of the user.

### CROSS-REFERENCE TO RELATED APPLICATION

This application is a continuation application, under 35 U.S.C. § 111 (a), of international application No. PCT/KR2024/016772, filed Oct. 30, 2024, which claims priority under 35 U. S. C. § 119 to Korean Patent Application No. 10-2023-0158419, filed Nov. 15, 2023, the disclosures of which are incorporated herein by reference in their entireties.

### BACKGROUND

### 1. Field

The present disclosure relates to a display apparatus and a method for measuring biometric information of the apparatus.

### 2. Description of Related Art

As functions of electronic apparatuses have become more advanced, development of biometric information measurement technologies using electronic apparatuses has been actively progressing. Recently, as demand for telemedicine has increased due to the risk of infection in physical hospitals, development of non-invasive biometric information measurement technologies that do not require physical contact has been actively progressing.

Accordingly, it has become possible to measure biometric information using a remote photoplethysmography (rPPG) method without physical contact with an electronic apparatus.

However, conventional biometric information measurement technologies have a limitation in that biometric information can be measured only when a user makes a request, and periodic measurement is difficult, thereby making long-term monitoring and health-condition analysis difficult.

### SUMMARY

A display apparatus according to an embodiment includes a display, a camera, at least one sensor, memory storing an artificial intelligence model trained based on learning data related to content viewing characteristics, and a processor.

The processor is configured to control the display to display content according to a user input, obtain data associated with a usage state of a user based on image data captured by the camera and sensing values of the at least one sensor, and based on the usage state being identified as suitable to measure biometric information based on the data associated with the usage state and the artificial intelligence model, newly obtain image data of the user through the camera, and obtain biometric information based on the newly obtained image data of the user.

A method for measuring biometric information of a display apparatus includes displaying content according to a user input, obtaining data associated with a usage state of a user based on a camera and at least one sensing value, based on an artificial intelligence model trained based on learning data related to content viewing characteristics and data associated with the usage state, identifying a state suitable to measure biometric information, and newly obtaining image data of the user through the camera, and obtaining biometric information based on the newly obtained image data.

In a non-transitory computer-readable recording medium storing a computer instruction that, when executed by a processor of a display apparatus, causes the display apparatus to perform operations, the operations include displaying content according to a user input, obtaining data associated with a usage state of a user based on a camera and at least one sensing value, based on an artificial intelligence model trained based on learning data related to content viewing characteristics and data associated with the usage state, identifying a state suitable to measure biometric information, and newly obtaining image data of the user through the camera, and obtaining biometric information based on the newly obtained image data.

### BRIEF DESCRIPTION OF DRAWINGS

FIG. 1 is a perspective view schematically illustrating a display apparatus according to at least one embodiment;

FIG. 2 is a block diagram provided to explain configuration of a display apparatus according to at least one embodiment;

FIG. 3 is a block diagram illustrating an example of detailed configuration of a display apparatus according to at least one embodiment;

FIG. 4 is a view provided to explain a biometric information measurement process of a display apparatus according to at least one embodiment;

FIG. 5 is a view provided to explain a biometric information measurement process through a camera of a display apparatus according to at least one embodiment;

FIGS. 6 and 7 are views provided to explain an rPPG method according to at least one embodiment;

FIGS. 8 and 9 are views provided to explain a biometric information providing process of a display apparatus according to at least one embodiment;

FIG. 10 is a view provided to explain an artificial intelligence model training process of a display apparatus according to at least one embodiment;

FIG. 11 is a flowchart of a method for measuring biometric information of a display apparatus according to at least one embodiment;

FIG. 12 is a view provided to explain an overall flow of biometric information measurement of a display apparatus according to at least one embodiment; and

FIG. 13 is a view illustrating a software structure for implementing embodiments of a display apparatus according to at least one embodiment.

### DETAILED DESCRIPTION

General terms that are currently widely used are selected as the terms used in the embodiments of the disclosure in consideration of their functions in the disclosure, but may be changed based on the intention of those skilled in the art or a judicial precedent, the emergence of a new technique, or the like. In addition, in a specific case, terms arbitrarily chosen by an applicant may exist, in which case, the meanings of such terms will be described in detail in the corresponding descriptions of the disclosure. Thus, the terms used in the embodiments of the disclosure need to be defined on the basis of the meanings of the terms and the overall contents throughout the disclosure rather than simple names of the terms.

In the disclosure, the expressions “have”, “may have”, “include” or “may include” used herein indicate existence of corresponding features (e.g., elements such as numeric values, functions, operations, or components), but do not exclude presence of additional features.

An expression, “at least one of A or/and B” should be understood as indicating any one of “A”, “B” and “both of A and B.”.

Expressions “first”, “second”, “1st,” “2nd,” or the like, used in the disclosure may indicate various components regardless of sequence and/or importance of the components, will be used only in order to distinguish one component from the other components, and do not limit the corresponding components.

When it is described that an element (e.g., a first element) is referred to as being “(operatively or communicatively) coupled with/to” or “connected to” another element (e.g., a second element), it should be understood that it may be directly coupled with/to or connected to the other element, or they may be coupled with/to or connected to each other through an intervening element (e.g., a third element).

A term of a singular number may include its plural number unless explicitly indicated otherwise in the context. It is to be understood that a term “include”, “formed of”, or the like used in the application specifies the presence of features, numerals, steps, operations, components, parts, or combinations thereof, mentioned in the specification, and does not preclude the presence or addition of one or more other features, numerals, steps, operations, components, parts, or combinations thereof.

In the disclosure, a “module” or a “unit” may perform at least one function or operation, and be implemented by hardware or software or be implemented by a combination of hardware and software. In addition, a plurality of “modules” or a plurality of “units” may be integrated into at least one module and be implemented by at least one processor (not shown) except for a ‘module’ or a ‘unit’ that needs to be implemented by specific hardware.

In this specification, a term ‘user’ may refer to a person using an electronic apparatus or an apparatus used by the person.

Hereinafter, an embodiment of the present disclosure will be described in greater detail with reference to the accompanying drawings.

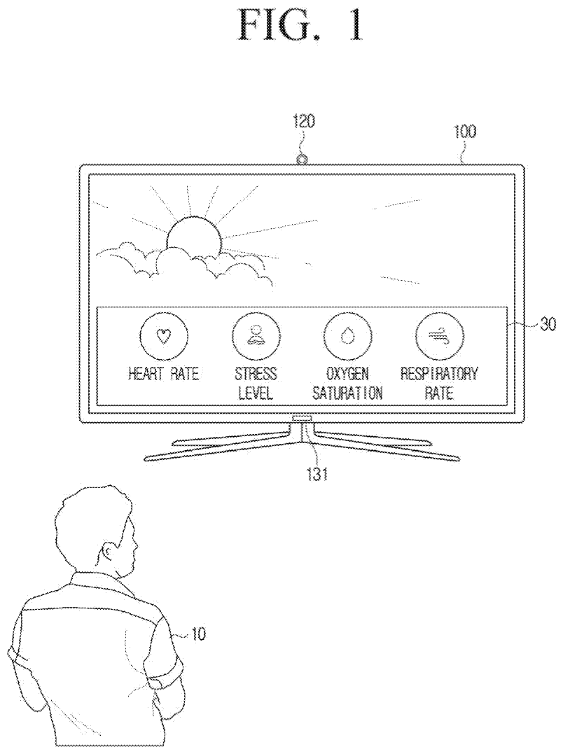

FIG. 1 is a perspective view schematically illustrating a display apparatus according to at least one embodiment.

A display apparatus 100 refers to an electronic apparatus that directly includes a display or that is connected to an external display (for example, a monitor). Specifically, the display apparatus 100 may be implemented as various devices, such as a monitor, a TV, a laptop PC, a PC, a kiosk, a mobile phone, a tablet PC, a refrigerator, and an air conditioner. When implemented in a form connected to an external display, the display apparatus 100 may also be referred to as an electronic apparatus or a terminal apparatus, but in the present disclosure, it is collectively referred to as the display apparatus 100.

Referring to FIG. 1, the display apparatus 100 may measure biometric information of a user 10. Specifically, the display apparatus 100 may capture the user 10 using a camera 120. The display apparatus 100 may measure biometric information of the user based on an image captured by the camera 120.

Biometric information refers to various types of information indicating biological characteristics of a person. Specifically, the biometric information may include heart rate, stress level, oxygen saturation, respiratory rate, heart rate variability, body temperature, and the like.

The display apparatus 100 may analyze the captured image to detect changes in skin color of the user 10, and may identify biometric information based on the detected result. In this case, a method for identifying biometric information may be a remote photoplethysmography (rPPG) method. The rPPG refers to a method for estimating various types of biometric information, such as a heart rate signal, by analyzing captured images of a person's face or body. A detailed description of the rPPG method will be provided later.

When biometric information is identified, the display apparatus 100 may provide the biometric information to the user. The biometric information may be provided in various ways. For example, when connected to the user's mobile phone or an external server, the display apparatus 100 may transmit the identified biometric information to the mobile phone or the external server.

Alternatively, when the display apparatus 100 directly includes a display as shown in FIG. 1, the display apparatus 100 may display the biometric information on the display. FIG. 1 illustrates a state in which a UI 30 including biometric information of the user 10 is displayed at a lower portion of the display. When the user selects an item on the UI, the display apparatus 100 may display the biometric information measured for the selected item. Accordingly, the user may easily check biometric information for various items.

When biometric information is measured based on captured images as described above, or when there are changes in a capturing environment or movement of the user, biometric information may not be measured or may be measured inaccurately.

For example, when ambient illuminance of an environment in which the user 10 and the display apparatus 100 are located is too bright or too dark, it may be difficult to detect changes in the user's skin color in the captured images, and thus measurement results may be inaccurate. Also, when the user speaks or moves, the measurement results may be inaccurate.

The display apparatus 100 according to at least one embodiment of the present disclosure may identify a usage state of the user by using captured images of the camera and sensing values of at least one sensor, and may first determine whether the user is in a state suitable for measuring biometric information based on the usage state. The usage state of the user may include a type of content being viewed, a viewing posture of the user, whether the user is speaking, whether the user is moving, a lighting condition, a viewing time, a viewing duration and the like. In other words, the usage state may include various states related to content viewing.

The display apparatus 100 may cumulatively manage usage states to extract a usage pattern. The usage pattern refers to information obtained by patternizing and organizing various types of information related to states in which the user ordinarily uses the display apparatus 100.

For example, when the display apparatus 100 is implemented as a TV for displaying content, the usage pattern may be organized based on various criteria such as a type of content, a content viewing time, personal characteristics of the user, age, and gender. Specifically, when the user watches or listens to music content during the daytime, may keep the normal lighting and sing along with the music or dance to it. In contrast, while the user watches or listens to music content during nighttime, the user may quietly enjoy the content. While the user watches content such as dramas or movies, the user may dim the lighting and gaze at the screen without speaking, regardless of the time. While the user watches content such as news, cultural, or educational content, the user may gaze at the screen without speaking while maintaining the normal lighting.

The display apparatus 100 may identify a state in which the user is gazing at the camera from the front without any particular movement or utterance and the ambient illuminance and a distance to the user are within an optimal range, as a state suitable for biometric information measurement.

While the user selects and views specific content, the display apparatus 100 may determine changes in the user's movement and whether the user is speaking based on captured images. In addition, the display apparatus 100 may sense brightness of an ambient environment using an illuminance sensor 131. The display apparatus 100 may identify changes in illuminance while the user selects and views specific content based on sensing values of the illuminance sensor 131. In other words, the display apparatus 100 may detect a pattern in which the user turns off a light when watching movie content.

The display apparatus 100 may identify and store a usage pattern of the user based on values sensed by the camera and at least one sensor over a preset time period.

The display apparatus 100 may determine whether a current state of the user is a state suitable for measuring biometric information based on the identified usage state, and when the state is suitable, may control the camera to capture the user and measure biometric information of the user based on the captured images.

As a result, the display apparatus 100 may autonomously determine a timing suitable for measuring biometric information even when the user 10 does not directly execute the display apparatus 100 or input a user manipulation.

For example, even when the user 10 does not directly input a command to check a heart rate, the display apparatus 100 may autonomously measure the heart rate of the user 10 and display the heart rate on the display.

Alternatively, even when the user 10 does not execute an application for measuring biometric information to check a stress index, the display apparatus 100 may autonomously determine a timing suitable for measuring biometric information, measure the stress index of the user, and display the stress index on the display.

Accordingly, since a state of the user may be measured during ordinary daily life of the user, sudden health risks may be detected in advance. In addition, biometric information of elderly persons or children who are not familiar with using devices may also be effectively measured.

Although the above description has explained a case in which biometric information is automatically measured, the present disclosure is not limited thereto, and the display apparatus 100 may measure biometric information of the user 10 even when a user manipulation is input.

In the above description, an embodiment in which the display apparatus 100 identifies a usage state of the user, determines whether a current state of the user is suitable for measuring biometric information, and performs an operation according to the determination result has been described, but an artificial intelligence model may be used to determine whether the user is in a state suitable for measuring biometric information.

For example, an artificial intelligence model trained based on learning data related to content viewing characteristics may be used. The display apparatus 100 may determine whether the user is in a state suitable for measuring biometric information based on the usage state of the user and the artificial intelligence model, and may perform an operation according to the determination result.

Hereinafter, a detailed description of an example in which the display apparatus 100 autonomously determines a timing suitable for measuring biometric information using an artificial intelligence model and measures biometric information of the user 10. Will be provided.

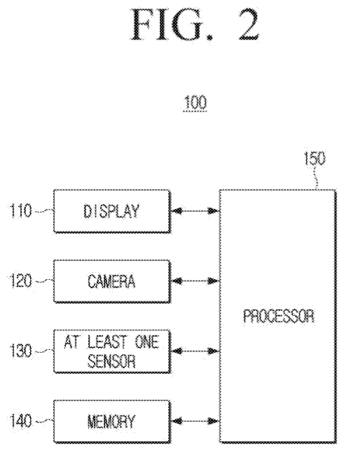

FIG. 2 is a block diagram provided to explain configuration of a display apparatus according to at least one embodiment.

Referring to FIG. 2, the display apparatus 100 may include a display 110, a camera 120, at least one sensor 130, memory 140, and a processor 150. However, the present disclosure is not limited thereto, and the display apparatus 100 may be implemented in a form in which some components are omitted or in a form in which additional components are included.

The display 110 is configured to display various screens such as content, biometric information, notification messages, and the like. The display 110 may be implemented in various types of displays such as a liquid crystal display (LCD), an organic light-emitting diode (OLED), a liquid crystal on silicon (LCoS), a digital light processing (DLP) display, a quantum dot (QD) display panel, a quantum dot light-emitting diode (QLED), a micro light-emitting diode (uLED), a Mini LED, or the like. Meanwhile, the display 110 may also be implemented as a touch screen combined with a touch sensor, a flexible display, a rollable display, a three-dimensional (3D) display, or a display in which a plurality of display modules are physically connected.

The camera 120 is configured to capture an object. Image data captured by the camera 120 may include both video and still images. Hereinafter, such data will be referred to as a captured image.

The camera 120 may include a lens and an image sensor. The lens may be a general-purpose lens, a wide-angle lens, a zoom lens, or the like, and may be determined according to the type, characteristics, and usage environment of the display apparatus 100. As the image sensor, a complementary metal-oxide semiconductor (CMOS) and a charge-coupled device (CCD) may be used.

The camera 120 in FIG. 1 may include at least one RGB camera. When the camera 120 is implemented as an RGB camera, the processor 150 may analyze biometric information of the user 10 using an rPPG method. Specifically, the processor 150 may extract R, G, and B pixel values of a region corresponding to the user's skin from a captured image captured by the RGB camera, and calculate changes in skin color based on changes in the extracted pixel values.

The camera 120 may operate in one of a plurality of operating states, such as a low-resolution state and a high-resolution state, under control of the processor 150. Meanwhile, the low-resolution state may be referred to as a low-resolution mode and the high-resolution state may be referred to as a high-resolution mode, and the present disclosure is not limited thereto, and they may also be referred to as a low-resolution scheme and a high-resolution scheme. However, in the present disclosure, they are described as a low-resolution state and a high-resolution state.

The camera 120 may be provided as a plurality of cameras, such as a low-resolution camera and a high-resolution camera. The low-resolution camera and the high-resolution camera may be selectively activated under control of the processor 150 to perform image capture. Here, activation includes being supplied with power and being switched to a state in which image capture is possible.

FIG. 1 illustrates a case in which one camera 120 selectively supports a low-resolution mode and a high-resolution mode.

In FIG. 2, the camera 120 is illustrated as being included in the display apparatus 100; however, the camera is not necessarily a built-in camera, and an external camera may be connected to and used with the display apparatus 100. The external camera may be connected through various input/output interfaces provided in the display apparatus 100, such as a USB port or an HDMI port. Alternatively, when the display apparatus 100 further includes a communicator, the display apparatus 100 may receive captured images from an external electronic apparatus including a camera.

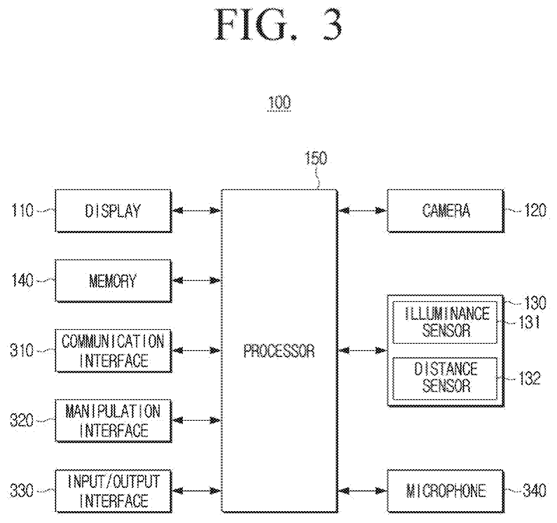

The at least one sensor 130 is configured to sense a surrounding state of the display apparatus 100 or a state of the user. The at least one sensor 130 may include an illuminance sensor 131 and a distance sensor 132. Detailed descriptions thereof will be provided with reference to FIG. 3.

The memory 140 may store at least one instruction, data, and program required for operation of the display apparatus 100. For example, the memory 140 may store an artificial intelligence model trained based on learning data related to content viewing characteristics. In addition, the memory 140 may store data on an illuminance range of an environment in which the user 10 is located.

The memory 140 may be implemented as embedded memory in the display apparatus 100 or as detachable memory, depending on the purpose of data storage. For example, data for driving the display apparatus 100 may be stored in embedded memory, and data for extension functions may be stored in detachable memory.

The memory embedded in the display apparatus 100 may be implemented as at least one of a volatile memory (e.g. a dynamic RAM (DRAM), a static RAM (SRAM), or a synchronous dynamic RAM (SDRAM)), or a non-volatile memory (e.g., a one-time programmable ROM (OTPROM), a programmable ROM (PROM), an erasable and programmable ROM (EPROM), an electrically erasable and programmable ROM (EEPROM), a mask ROM, a flash ROM, a flash memory (e.g. a NAND flash or a NOR flash), a hard drive, or a solid state drive (SSD)).

The memory 140 may be implemented as single memory that stores data generated in various operations according to the present disclosure; however, the present disclosure is not limited thereto, and the memory 140 may be implemented to include a plurality of memories that respectively store different types of data or data generated in different stages.

The processor 150 is configured to control the operations of the display apparatus 100. The processor 150 may be implemented as a digital signal processor (DSP) or a microprocessor. However, the processor 150 is not limited thereto, and the processor 150 may include, or be defined as, one or more of a central processing unit (CPU), a micro controller unit (MCU), a micro processing unit (MPU), a controller, an application processor (AP), a communication processor (CP), an ARM processor, and an artificial intelligence (AI) processor. In addition, the processor 150 may also be implemented as a system on chip (SoC) or a large scale integration (LSI) with a built-in processing algorithm, or may be implemented in the form of a field programmable gate array (FPGA). The processor 150 may perform various functions by executing computer executable instructions stored in the memory 140.

The processor 150 may be implemented as a single core processor including a single core, or as one or more multicore processors including a plurality of cores (e.g., homogeneous multicore or heterogeneous multicore). When the one or more processors 150 are implemented as a multicore processor, each of the plurality of cores included in the multicore processor may include internal memory of the processor, such as cache memory and an on-chip memory, and a common cache shared by the plurality of cores may be included in the multicore processor. Each of the plurality of cores (or some of the plurality of cores) included in the multi-core processor may independently read and perform program instructions to implement the method according to an embodiment, or all (or some) of the plurality of cores may be coupled to read and perform program instructions to implement the method according to an embodiment.

The processor 150 may control the display 110 to display content according to a user input. The processor 150 may identify a usage state of the user 10 by using image data of the camera 120 and sensing values of the at least one sensor 130. The processor 150 may obtain data on the user's usage state based on the image data of the camera 120 and the sensing values of the at least one sensor 130. The processor 150 may identify whether the user 10 is in a state suitable for measuring biometric information of the user 10 through an artificial intelligence model trained based on learning data related to the identified usage state and content viewing characteristics.

When the state is identified as a state suitable for measuring biometric information, the processor 150 may newly obtain image data of the user through the camera 120 and obtain biometric information based on the obtained data of the user. The processor 150 may measure biometric information of the user 10 based on the obtained image data.

The processor 150 may measure biometric information when an event occurs. Here, the event may include an event in which a preset time period elapses, an event in which a preset time elapses after the display apparatus 100 is turned on, an event in which a preset time elapses after a user commands content output or changes a type or a providing source of content being viewed, illuminance, or the like, an event in which illuminance changes, and an event in which a viewing duration, a usage time period, a device usage time, or a content/app session maintenance time changes. The event may also be expressed as an occurrence or an activity, but is referred to as an event in the present disclosure. In addition, in the present disclosure, content may include various types, such as terrestrial broadcast content, cable broadcast content, satellite content, a PC screen, a web page screen provided by a web server, a playback screen of a multimedia playback device, and a game execution screen of a game player. A providing source may also be variously implemented, such as a broadcasting station, a cable broadcasting station, a satellite antenna, a PC, a multimedia playback device, a game player, and a set-top box.

When it is determined that at least one of the above-described events has occurred, he processor 150 may automatically measure biometric information of a user.

Specifically, when the user 10 is viewing content through the display apparatus 100, the processor 150 may measure biometric information of the user 10 using the camera 120 and the at least one sensor 130. The processor 150 may use an artificial intelligence model to determine the biometric information of the user 10.

The artificial intelligence model may be a model that has been pre-trained based on learning data related to content viewing characteristics. A manufacturer of the display apparatus 100 or other related entities may collect image data obtained by capturing states of users who view various types of content in advance, and may label various items, such as appearances of the users included in the captured image data, types of content, viewing time, and lighting conditions, to thereby obtain a large-scale data set. The manufacturer of the display apparatus 100 or other related entities may train the artificial intelligence model by inputting the obtained data set into the artificial intelligence model and feeding back analysis results of the artificial intelligence model.

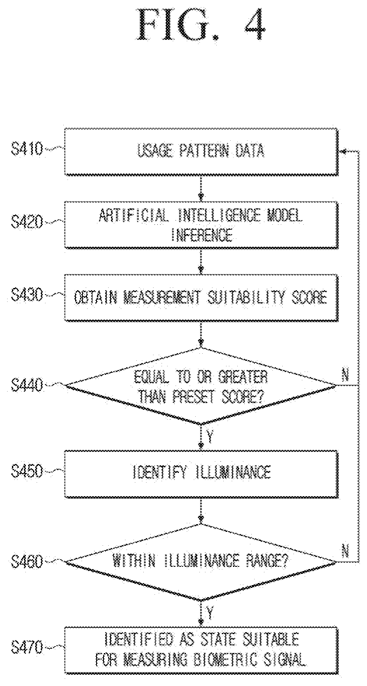

The processor 150 may determine whether a current state of the user 10 is suitable for measuring biometric information based on a usage state of the user 10 who is viewing content and the above-described artificial intelligence model. A specific determination method will be described with reference to FIG. 4.

When it is determined that the state of the user 10 is suitable for measuring biometric information, the processor 150 may drive the camera 120 in a low-resolution state and a high-resolution state to capture the user 10.

Meanwhile, when it is determined that the state of the user 10 is suitable for measuring biometric information, the processor 150 may newly obtain captured image data of the user through the camera 120 and may obtain biometric information based on the obtained captured image data. In addition, the processor 150 may obtain biometric information of the user 10 by reusing camera image data used to obtain the above-described data on the usage state. Specifically, the processor 150 may obtain data on the usage state of the user 10 through the camera 120 and may store the camera image data used at that time in the memory 140. Thereafter, when it is determined that the state is suitable for measuring biometric information, the processor 150 may obtain biometric information of the user 10 by reusing the image data stored in the memory 140.

The processor 150 may determine a movement state of the user 10 based on captured images obtained in the low-resolution state. Specifically, the processor 150 may divide all pixels included in each of a plurality of consecutively captured image frames obtained in the low-resolution state into a plurality of blocks each composed of n by m pixels. The processor 150 may detect a representative value representing features of pixels in each block. The representative value may be an average pixel value of pixels in each block, but is not limited thereto, and may be a maximum pixel value, a minimum pixel value, or a Root Means Square (RMS) value.

The processor 150 may detect an edge of an object included in a captured image by connecting blocks that have representative values within a similar range and are disposed at continuous positions among a plurality of blocks. The processor 150 may identify a size of the corresponding object based on a number of blocks included in the edge. Further, the processor 150 may identify a shape of the corresponding object based on a shape of the edge and accordingly identify a type of the corresponding object.

For a user, the processor 150 may identify a state corresponding to the shape of the edge using a database in which shapes for various states, such as a standing state, a lying state, and a sitting state of a user, are classified and stored in advance. When a user is recognized, the processor 150 may compare blocks within the edge corresponding to the user in a plurality of consecutively captured image frames and check changes in the number and positions of the blocks. When a difference equal to or greater than a preset error range is identified as a result of the checking, the processor 150 may determine that the user has moved. When operations are performed based on images captured in the low-resolution state, a computational burden may be reduced because the number of pixels is not large.

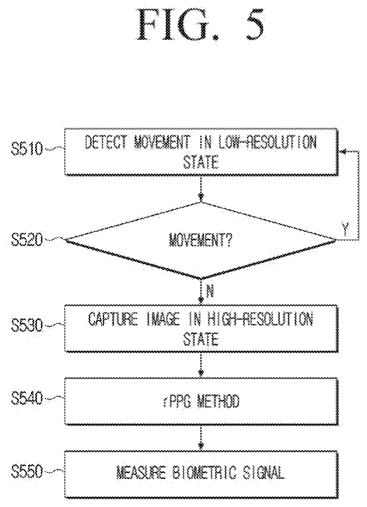

When movement of the user 10 is not detected for a preset time, the processor 150 may drive the camera 120 in the high-resolution state. The processor 150 may measure biometric information of the user 10 based on captured images obtained in the high-resolution state. A specific measurement method will be described with reference to FIG. 5.

FIG. 3 is a block diagram illustrating an example of detailed configuration of a display apparatus according to at least one embodiment.

According to FIG. 3, the display apparatus 100 may further include a communication interface 310, a manipulation interface 320, an input/output interface 330, and a microphone 340, in addition to the display 110, the camera 120, the sensor 130, the memory 140, and the processor 150. Among the components of FIG. 3, descriptions of parts that are the same as those described in FIG. 2 regarding the display 110, the camera 120, the memory 140, and the processor 150 are omitted to avoid redundancy.

The communication interface 310 is configured to perform communication with at least one external apparatus. The communication interface 310 may include at least one wireless communication module, at least one wired communication module, and the like. Each communication module may be implemented in the form of at least one hardware chip. As one example, the processor 150 may transmit measured biometric information or the like to an external apparatus through the communication interface 310.

As another example, when it is identified that the user is in a dangerous state based on the measured biometric information, the processor 150 may transmit a danger alert signal to an external apparatus through the communication interface 310. Specifically, when a change in a heart rate of the user is not detected for a preset time or is detected to be above a normal range or below a normal range, the processor 150 may identify the user as being in the dangerous state. The processor 150 may transmit the danger alert signal to a server apparatus or a terminal apparatus operated by a hospital, a police station, an emergency rescue center, or a fire station, or may transmit the danger alert signal to a terminal apparatus of a pre-registered guardian. To this end, the memory 140 may store a telephone number, an e-mail address, a messenger ID, and the like of a recipient that is to receive a danger alert signal.

The manipulation interface 320 is configured to receive a user manipulation. The manipulation interface 320 may include various buttons, a touch screen, and the like provided on a main body of the display apparatus 100. However, the manipulation interface 320 is not limited thereto, and may also be implemented by another electronic apparatus such as a remote controller. The user 10 may select a desired menu through the manipulation interface 320. In addition, the user 10 may input a user manipulation for setting a biometric information measurement period or the like through the manipulation interface 320. In FIG. 3, the manipulation interface 320 and the display 110 are illustrated as separate components, but when the manipulation interface 320 is implemented as a touch screen, it may be integrally formed with the display 110.

The input/output interface 330 is configured to input and output various external signals. The input/output interface 330 may receive at least one of audio signals and image signals from various content sources (for example, a web server, a media player, and a user terminal apparatus). In addition, the input/output interface 330 may transmit and receive data or control signals with various external apparatuses (for example, another display apparatus, a remote controller, a mobile phone, a speaker, a set-top box, a television, lighting equipment, etc.). The input/output interface 330 may be implemented as at least one wired input/output interface among High Definition Multimedia Interface (HDMI), Mobile High-Definition Link (MHL), Universal Serial Bus (USB), USB Type-C (USB-C), DisplayPort (DP), Thunderbolt, a Video Graphics Array port (VGA), an RGB port, D-subminiature (D-SUB), and Digital Visual Interface (DVI). As described above, when an external camera, rather than an internal camera, is connected and used, the external camera may be connected through the input/output interface 330.

Meanwhile, the at least one sensor 130 includes an illuminance sensor 131, a distance sensor 132, and the like.

The illuminance sensor 131 is configured to sense illuminance around the display apparatus 100. The illuminance sensor 131 may measure light intensity using a photoelectric effect. The photoelectric effect refers to a phenomenon in which, when light having a frequency equal to or higher than a specific frequency is incident on a metal, electrons are generated by light energy and a current flows. The processor 150 may identify an illuminance around the display apparatus 100 based on a sensing value of the illuminance sensor 131.

For identifying a usage state of the user, the processor 150 may also use illuminance sensed during content viewing. For example, an illuminance value measured during viewing of a drama or a movie may be included in data on the usage state and stored.

In addition, when the identified illuminance is included in an illuminance range stored in the memory 140, the processor 150 determines that the state is suitable for measuring biometric information. Detailed descriptions thereof will be provided with reference to FIG. 4.

The distance sensor 132 is configured to sense a distance to an external object. The processor 150 may identify a distance between the display apparatus 100 and the user 10 based on a sensing value of the distance sensor 132. The distance sensor 132 may include at least one of an ultrasonic sensor, an infrared sensor, a laser sensor, an optical distance sensor, a radar sensor, a Lidar sensor, a photodiode sensor, or a time of flight sensor. The processor 150 may further check whether the user is in a state suitable for measuring biometric information based on the identified distance.

As one example, when the user 10 is positioned too close to the display apparatus 100 or too far away from the display apparatus 100, the processor 150 may determine that accurate biometric information measurement is difficult.

When there is difficulty in accurately measuring biometric information, the processor 150 may postpone measuring biometric information. According to another embodiment, the processor 150 may display, on the display 110, a notification or a message for causing a user to adjust a distance.

The microphone 340 is configured to receive various audio signals. The microphone 340 may receive a voice of the user or other sounds and provide them to the processor 150. When a voice of the user is input through the microphone 340, the processor 150 may determine that the user is in a speaking state and may identify the state as unsuitable for measuring biometric information. On the other hand, when a voice of the user is not input for a preset time or longer, the processor 150 may determine that the user is not speaking and may identify the state as suitable for measuring biometric information.

As described above, the display apparatus 100 may include various components. Accordingly, the display apparatus 100 may perform various operations together to measure biometric information of the user 10.

FIG. 4 is a view provided to explain a biometric information measurement process of a display apparatus according to at least one embodiment.

Referring to FIG. 4, the processor 150 calculates data on a usage state identified by the camera 120 and the at least one sensor 130 in a state in which the above-described artificial intelligence model is executed (S410). The data on the usage state may include data on at least one among various items, such as a type of content being viewed by a user, a viewing posture of the user, whether the user is speaking, whether the user is moving, a lighting condition, a viewing time, and a viewing duration.

The processor 150 performs inference by using the data on the usage state as an input value of the artificial intelligence model (S420). The processor 150 may input data regarding a user's viewing posture to an artificial intelligence model in a form of an identification value individually set for a standing posture, a sitting posture, and a lying posture (for example, a digital value in which 0 and 1 are combined), or may input the data to the artificial intelligence model in a form of a captured image obtained by capturing the user.

The inference refers to a process in which, after the artificial intelligence model is trained, the artificial intelligence model derives an answer by performing prediction, classification, inference, and the like for new input data. Subsequently, the processor 150 may obtain a measurement suitability score output from the artificial intelligence model (S430).

The measurement suitability score refers to information that quantifies a degree to which a usage state of the user 10 is suitable for measurement. The measurement suitability score may be variously referred to as a usage pattern score, a characteristic score, a feature score, or a target score; however, in the present disclosure, it is referred to as a measurement suitability score.

The measurement suitability score may be expressed in units of %, but is not necessarily limited thereto. The measurement suitability score may be a predicted value for a reliability score of biometric information based on signal-to-noise ratio (SNR) information to be described later. Detailed descriptions thereof will be provided later.

As one example, when the user 10 is viewing news in an evening time period, the processor 150 performs inference by using data such as a viewing genre, a used app, a viewing duration, and a viewing time period as input values of the artificial intelligence model. As in the above-described example, when a user normally watches news during an evening time period while quietly viewing under a normal lighting condition without significant movement or speaking, an artificial intelligence model may output a high value of a measurement suitability score.

Although the artificial intelligence model is a model trained in advance based on learning data related to various content viewing characteristics as described above, the processor 150 may additionally further train the artificial intelligence model by using the user's usage pattern.

Specifically, while performing inference through the artificial intelligence model by using data on the usage state of the user 10 as input values, the processor 150 stores the data on the usage state in the memory 140. The processor 150 accumulates the usage state at preset time intervals to analyze a usage pattern, and may train the artificial intelligence model by using data on the usage pattern as learning data.

When the obtained measurement suitability score is equal to or greater than a preset score and illuminance identified based on a sensing value of the illuminance sensor 131 among the at least one sensor is included within an illuminance range stored in the memory 140, the processor 150 may identify a state as a state suitable for measuring biometric information.

When the obtained measurement suitability score is equal to or greater than a preset score (S440), the processor 150 may identify illuminance based on a sensing value of the illuminance sensor 131 among the at least one sensor (S450). On the contrary, when the measurement suitability score does not exceed the preset score, the processor 150 may not perform biometric information measurement. In this case, the processor 150 may add the case in which the measurement stability score does not exceed the preset score to usage pattern data stored in the memory 140 to update learning data.

For example, when a preset score is 0.7, the processor 150 may, in a case where a calculated measurement suitability score is equal to or greater than 0.7, drive the illuminance sensor 131 to obtain a sensing value, and may identify illuminance based on the sensing value.

When the identified illuminance is included within the illuminance range stored in the memory 140 (S460), the processor 150 may determine that the user 10 is in a state suitable for measuring biometric information (S470). On the contrary, when the identified illuminance is not included within the illuminance range, biometric information measurement is not performed.

As one example, when the illuminance range is set to 300 lx to 500 lx and the illuminance measured using the illuminance sensor 131 is 400 lx, the processor 150 may determine that the state is suitable for measuring biometric information. A manufacturer of the display apparatus 100 or a related company may repeatedly perform a task of comparing results of measuring biometric information using captured images captured under various illuminances, set an illuminance range in which accurate biometric information measurement is possible, and store the illuminance range in the memory 140. Information on the illuminance range may be updated at any time or periodically.

Meanwhile, the processor 150 may calculate an illuminance score representing each illuminance range by classifying the illuminance measured using the illuminance sensor 131 according to preset illuminance ranges. Specifically, the processor 150 may calculate the illuminance score in a manner as shown in the following table.

TABLE 1

| Illuminance (lx) | Illuminance score |
|---|---|
| 0~100 | 0.5 |
| 100~200 | 0.6 |
| 200~300 | 0.7 |
| 300~400 | 0.8 |
| 400~500 | 0.8 |
| 500~600 | 0.7 |

According to Table 1, when measured illuminance is 100 lx to 200 lx, the processor 150 may calculate an illuminance score as 0.6, and when the measured illuminance is 300 lx to 400 lx, the processor 150 may calculate the illuminance score as 0.8. The processor 150 may update the illuminance score by assigning an additional score to the illuminance score according to a reliability score that is to be calculated later. In other words, even when the measured illuminance is included within an illuminance range that is set as being suitable for measurement, if the reliability score that is to be calculated later is calculated to be low, the illuminance range may be adjusted and the illuminance score may be updated. Conversely, when the reliability score is calculated to be high, an additional score may be assigned to the illuminance score. Table 2 shows an example of results of updating the illuminance score based on the reliability score.

TABLE 2

| Illuminance (lx) | Illuminance score |
|---|---|
| 0~100 | 0.5 |
| 100~200 | 0.6 |
| 200~300 | 0.7 |
| 300~400 | 0.85 |
| 400~500 | 0.8 |
| 500~600 | 0.7 |

When the reliability score in a specific illuminance range is equal to or greater than a specific value, the processor 150 may give an additional score to the corresponding illuminance range. For example, when the reliability score is equal to or greater than 0.9 in the 300-400 lx range, the processor 150 gives an additional score of 0.05 to update the illuminance score to 0.85. On the other hand, when the reliability score is equal to or less than 0.7, the processor 150 gives an additional score of −0.05 to update the illuminance score to 0.75. Table 2 shows a case in which the reliability score is 0.9. As described above, the processor 150 may convert the illuminance range into the form of an illuminance score and update and use the same according to a reliability score. The processor 150 may recognize that the illuminance environment is suitable for measuring biometric information when the updated illuminance score is equal to or greater than a specific value for measurement. In this manner, by using the updated illuminance score, it is possible to increase prediction performance of a measurement time point.

Hereinafter, a process of measuring biometric information in the processor 150 when it is determined that a state is suitable for measuring biometric information will be described.

FIG. 5 is a view provided to explain a biometric information measurement process through a camera of a display apparatus according to at least one embodiment.

Referring to FIG. 5, when the processor 150 identifies that the user 10 is in a state suitable for measuring biometric information, the processor 150 may detect a movement of the user 10 based on a captured image obtained through the camera 120 in a low-resolution state (S510).

The low-resolution state refers to a state in which the number of pixels that sense light in an image sensor of the camera 120 is reduced, so that a captured image is generated at a relatively low resolution.

When capturing is performed in the low-resolution state, it is possible to identify whether the user is moving at a level at which the user's appearance is not clearly represented, thereby protecting the user's privacy.

When there is a large amount of movement of the user 10, the processor 150 waits without performing measurement.

On the other hand, when the movement of the user is not detected for a preset time (S520), the processor 150 may obtain biometric information of the user 10 based on a captured image obtained through the camera 120 in a high-resolution state.

The processor 150 may drive the camera 120 in the high-resolution state to capture the user 10 (S530). The high-resolution state refers to a state in which the number of pixels sensing light in the image sensor is increased compared to the low-resolution state, so that the user's face or body part can be clearly captured.

Although a case in which one camera is selectively driven in the low-resolution state and the high-resolution state has been described above, when both a low-resolution camera and a high-resolution camera are provided, each camera may be sequentially used.

The processor 150 may identify at least one face region or another body part from a captured image obtained in the high-resolution state. The processor 150 may measure biometric information of the user 10 from the identified face region by using a remote photoplethysmography (rPPG) method (S540) (S550).

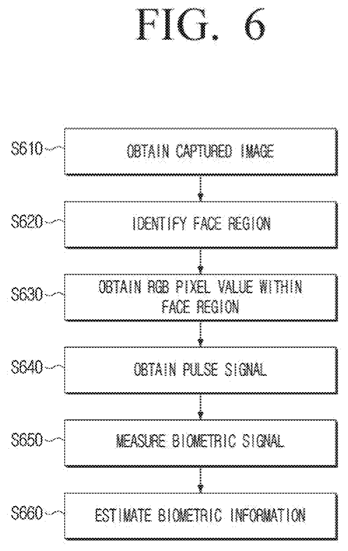

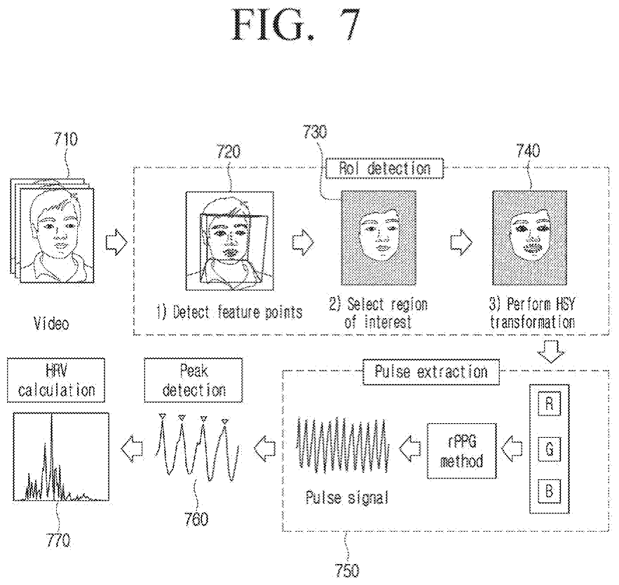

FIGS. 6 and 7 are views provided to explain an rPPG method according to at least one embodiment.

Referring to FIG. 6, the processor 150 may obtain a captured image captured in the high-resolution state (S610). The processor 150 may identify a face region in a plurality of consecutive image frames of the captured image (S620).

Specifically, the processor 150 divides all pixels included in each of a plurality of consecutive image frames captured in a high-resolution state into a plurality of blocks each composed of n*m pixels, and detects a representative value for each block. The processor 150 may detect an edge by connecting a plurality of blocks that are positioned continuously with each other and have representative values within a similar range among the blocks in one image frame. Examples of the representative values and the edge detection method have been described above and thus, a duplicate description will be omitted.

When the camera 120 is installed at a central portion of the display apparatus and a user is viewing the display apparatus 100, a front face of the user may be included in a captured image. Accordingly, the user's face may have a circular or vertically elliptical shape. When a connection form of blocks corresponding to an edge is a circular shape or a vertically elliptical shape, the processor 150 may determine that blocks inside the edge correspond to the user's face. The processor 150 may further extract blocks corresponding to the user's eyes, nose, and mouth within the blocks corresponding to the user's face, thereby identifying overall facial characteristics of the user. The processor 150 may detect R, G, and B pixel values of respective pixels in blocks corresponding to a region of the user's face in which heart-rate changes are well represented (S630). The processor 150 may extract a pulse signal according to a change state of at least one of the detected R, G, and B pixel values by using the rPPG method (S640).

The rPPG method is a method of measuring biometric information based on capturing a face region of a person located at a certain distance from a camera and extracting minute movements from a captured image. In other words, it refers to a non-invasive method of remotely measuring heart rate and blood flow information.

Specifically, when changes in R, G, and B pixel values of pixels constituting the same facial portion in a plurality of consecutively captured image frames are measured, a signal in the form of a pulse signal may be extracted.

The processor 150 may obtain R, G, and B pixel values of each pixel in a face region identified in a plurality of consecutive image frames among captured images obtained in the high-resolution state, obtain a pulse signal according to a change state of at least one of the obtained R, G, and B pixel values by using the rPPG method, and obtain biometric information of the user based on changes in magnitudes of peaks of the obtained pulse signal.

The processor 150 may detect peaks of the pulse signal and measure biometric information based on magnitudes of the detected peaks and a peak occurrence period, and the like (S650). For example, depending on a change in heart rate, a change cycle of color or a color value of some regions of the user's face may change. Also in the case of body temperature, as the body temperature increases, an R value may be measured more strongly. When the user becomes pale due to poor blood circulation, magnitudes of all of R, G, and B values may be measured to be large. The processor 150 estimates biometric information according to such changes of the R, G, and B pixel values (S660).

FIG. 7 is a view provided to explain a process of measuring heart rate variability (HRV) among a user's biometric information by an rPPG method. Prior to biometric information measurement using the rPPG method, the processor 150 may capture a face region of the user with a camera in a high-resolution state to obtain a captured image (710).

The processor 150 may identify, for each of a plurality of consecutive image frames in the captured image, a landmark or a representative value of the face region on a pixel basis or a pixel block basis (720). The processor 150 performs a process of selecting a region of interest (ROI) based on the identified landmark, and may remove eye and mouth regions using the above-described landmark. The processor 150 may convert each frame into a Hue, Saturation, Value (HSV) color space to extract a skin-color region, and remove hair and beard regions (740). Here, the HSV refers to a color space that represents hue, saturation, and value, in contrast to the RGB color space.

The processor 150 may extract a pulse signal from color changes of the skin-color region (750). Prior to applying the rPPG method, the processor 150 may calculate a spatial average of pixel values of the skin-color region and decompose the same into RGB components. The processor 150 may extract a pulse signal through the rPPG method using the decomposed RGB components.

The processor 150 may convert the pulse signal into a frequency domain. The processor 150 may detect a peak corresponding to a frequency component of a heart beat based on the pulse signal converted into the frequency domain (760). The processor 150 may track the detected peak to calculate a heart rate and obtain a heart rate variability measurement value (770).

The processor 150 may measure biometric information of the user 10 using the above-described rPPG method.

Meanwhile, the processor 150 may obtain signal-to-noise ratio (SNR) information in order to measure reliability of biometric information measured using the rPPG method. The SNR refers to a value representing a signal-to-noise ratio and is expressed in decibels. As the SNR value increases, reliability of a measurement result may increase.

The processor 150 may update a measurement suitability score according to a reliability score for the measured biometric information obtained based on the SNR information calculated from the measured biometric information.

The processor 150 may determine quality and reliability of a measurement result based on the SNR information of biometric information measured using the rPPG method, and quantify the same to calculate a reliability score.

The processor 150 may distinguish, in an rPPG signal, a signal having a magnitude less than a threshold (that is, noise) and a signal having a magnitude equal to or greater than the threshold, and calculate a ratio thereof to convert the same into a reliability score.

There are various methods of calculating the reliability score using the SNR information. In the present disclosure, a final score may be calculated by linearly mapping the same to a value between 0 and 1. However, the method of calculating reliability using the SNR information is not limited thereto and may be calculated in various ways.

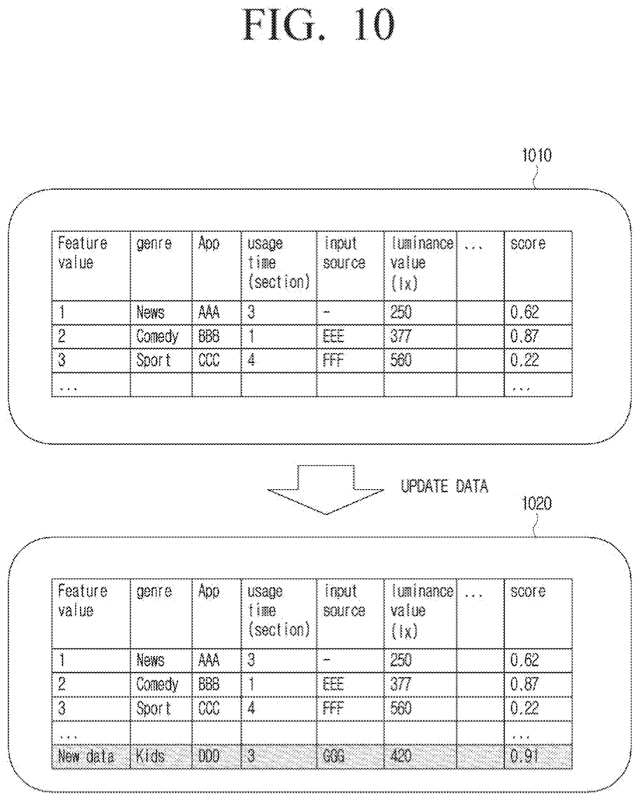

The processor 150 may update the above-described measurement suitability score according to the calculated reliability score. Details thereof will be described with reference to FIG. 10.

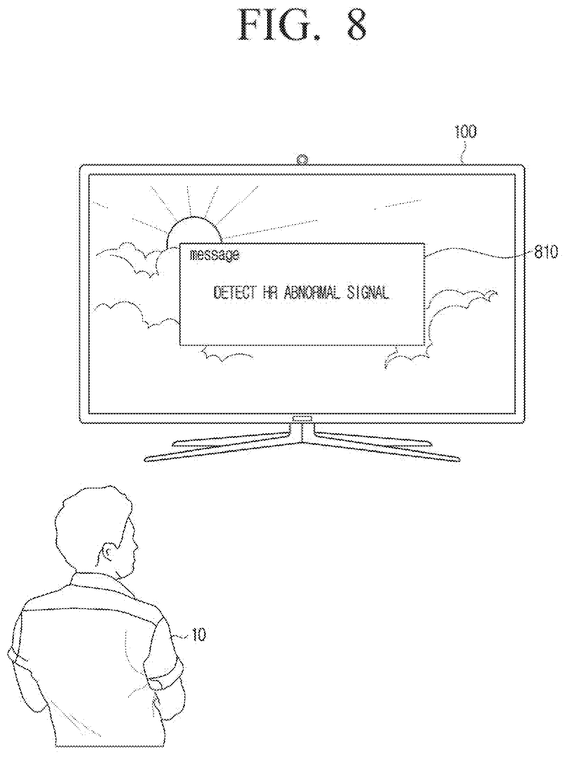

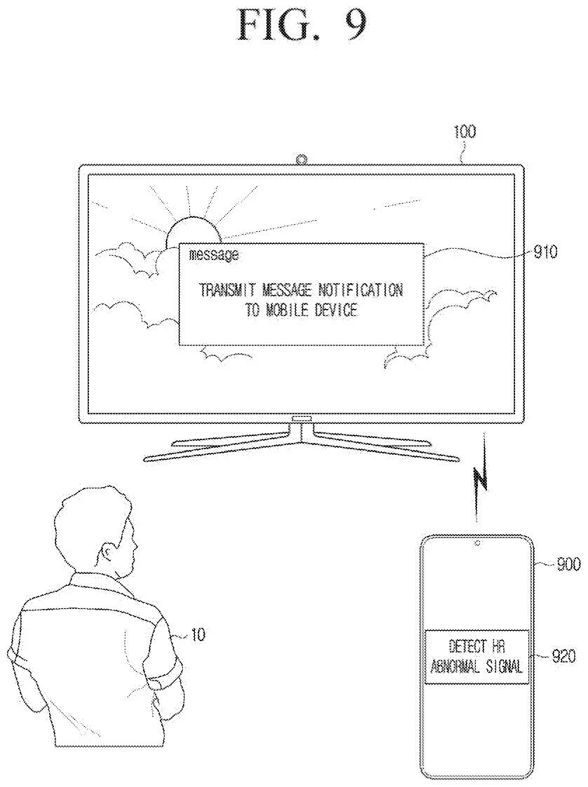

FIGS. 8 and 9 are views provided to explain a biometric information providing process of a display apparatus according to at least one embodiment.

Referring to FIG. 8, the processor 150 automatically determines timing suitable for measuring biometric information, measures biometric information of a user, and stores the same in the memory 140. The processor 150 may analyze the biometric information stored in the memory 140 on a daily, monthly, and yearly basis and provide the same to the user in the form of graphs for each biometric information. When the processor 150 measures biometric information of the user 10, if the biometric information is measured to be worse than previously measured biometric information or a preset normal range, the processor 150 may notify an abnormal signal detection message on the display 110 (810).

For example, when the display apparatus 100 measures a heart rate among biometric information of the user 10, and a heart rate value different from resting heart rate data of the user 10 is measured, the display apparatus 100 may provide a message such as heart rate (HR) abnormal signal detection on the display 110, or may notify the user 10 of an abnormal signal detection result through a speaker or the like.

FIG. 9 illustrates a case in which the display apparatus 100 that measures biometric information notifies another external electronic apparatus of a measurement result. According to FIG. 9, when biometric information characteristics become worse compared to previous ones, or when biometric information differs from a preset normal range by a predetermined amount or more, the display apparatus 100 may provide a biometric information abnormal signal detection result of the user 10 to a mobile device 900 connected to the display apparatus 100 (910, 920).

For example, when the user 10 falls asleep while viewing content or uses a mobile device while viewing content, the display apparatus 100 may provide a biometric information abnormal signal detection result of the user 10 through a notification or a message via the mobile device 900 linked with the display apparatus 100.

As another example, the user 10 may receive information on biometric information measured through the display apparatus 100 using the mobile device 900.

In FIG. 9, a case in which a message 920 is transmitted to the mobile device 900 owned by the user 10 of the display apparatus 100 is illustrated, but as described above, the measurement result or such a notification message may also be transmitted to another external terminal device or a server apparatus. Accordingly, when it is identified that the user has collapsed or has a health abnormality, rescue can be performed immediately or a guardian can be notified.

FIG. 10 is a view provided to explain an artificial intelligence model training process of a display apparatus according to at least one embodiment.

The processor 150 may update learning data by using data on a user state identified through the camera 120 and the at least one sensor 130. The processor 150 may further train an artificial intelligence model using the updated learning data. The processor 150 may update a measurement suitability score according to a reliability score.

Referring to FIG. 10, updated learning data 1020 is shown in which new data is added to existing learning data 1010. Specifically, a case is shown in which user state data indicating that the user watched children's content (Kids) provided by a DDD app for three hours and that an illuminance value during viewing was 420 is included. As described above, the learning data may include data on a plurality of content viewing characteristics obtained by combining at least one of content type, content providing source, viewing duration, viewing time, and illuminance level, and data on a plurality of measurement suitability scores set for each of the plurality of viewing characteristics. However, the learning data is not limited thereto, and may further include data on viewing characteristics.

For example, when biometric data is measured while the user 10 is viewing the kids genre using the display apparatus 100, user pattern data such as content type, viewing duration, viewing time, illuminance, and measurement suitability score may be added to learning data of the artificial intelligence model (1020).

On the other hand, when biometric data is measured while the user is viewing the news content already included in existing learning data, the existing learning data may be updated, and the measurement suitability score may be updated according to a newly calculated reliability score (1020).

As such, the display apparatus 100 newly measures biometric information of the user 10, and in this process, adds user pattern data according to new user state characteristics and reliability scores to the artificial intelligence model to add or update learning data, and may add or update a measurement suitability score according to the reliability scores.

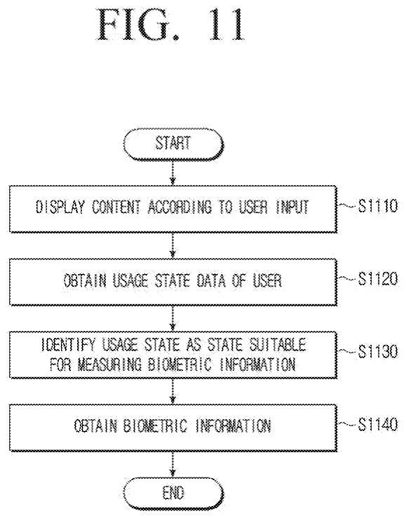

FIG. 11 is a flowchart of a method for measuring biometric information of a display apparatus according to at least one embodiment.

Referring to FIG. 11, the display apparatus displays content according to a user input (S1110). The display apparatus obtains data on a user's usage state based on a camera and at least one sensing value (S1120). The display apparatus identifies a state suitable for measuring biometric information based on an artificial intelligence model trained based on learning data related to content viewing characteristics and the identified user state (S1130). The display apparatus newly obtains captured data of the user through the camera, and obtains biometric information based on the obtained captured data (S1140).

A specific method of determining whether a state is suitable for measuring biometric information and measuring biometric information has been described in detail in the various embodiments described above, and thus a duplicate description will be omitted.

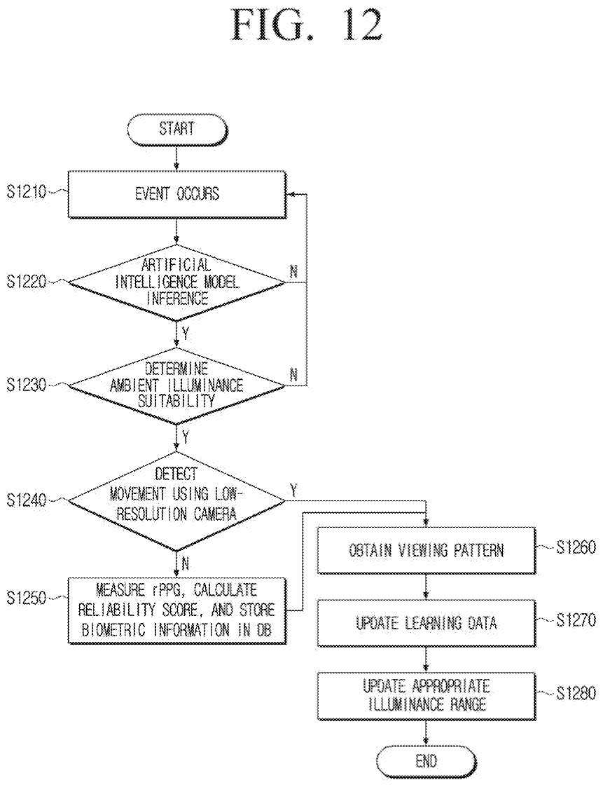

FIG. 12 is a view provided to explain an overall flow of biometric information measurement of a display apparatus according to at least one embodiment.

Referring to FIG. 12, when an event for measuring biometric information occurs (S1210), the display apparatus executes an artificial intelligence model to obtain a measurement suitability score (S1220). When the measurement suitability score is equal to or greater than a threshold score, the display apparatus determines illuminance suitability (S1230). When illuminance suitability is determined, the display apparatus detects whether there is movement using a camera in a low-resolution state (S1240). When no movement is detected, the display apparatus measures biometric information through an rPPG method using a camera in a high-resolution state and calculates a reliability score (S1250).

Subsequently, the display apparatus detects a usage pattern based on data on a user's usage state (S1260), updates learning data based on the usage pattern (S1270), and updates an illuminance range based on an illuminance score (S1280).

The operations and methods described in the various flowcharts above may be performed by a display apparatus having the configuration illustrated in FIGS. 2 and 3, but are not necessarily limited thereto, and may also be performed by an electronic apparatus having various configurations.

Meanwhile, the operations and methods according to the various embodiments described above may be performed according to execution of an artificial intelligence model and other software modules.

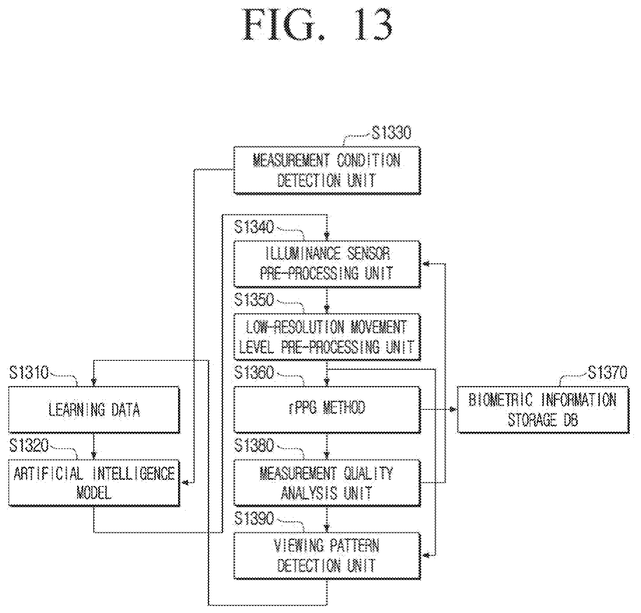

FIG. 13 is a view illustrating a software structure for implementing embodiments of a display apparatus according to at least one embodiment.

According to FIG. 13, memory of the display apparatus 100 may store a learning data module S1310, an artificial intelligence model module S1320, a measurement condition detection module S1330, an illuminance sensor preprocessing module S1340, a low-resolution movement level preprocessing module S1350, an rPPG method module S1360, a biometric information storage database module S1370, a measurement quality analysis module S1380, and a viewing pattern detection module S1390. However, the memory is not limited thereto, and may further include other modules. The processor 150 may execute each software module to perform operations according to the various embodiments described above.

The learning data module S1310 and the artificial intelligence model module S1320 calculate a measurement suitability score using an artificial intelligence model based on user learning data as described in detail above, and thus duplicate description will be omitted.

The measurement condition detection module S1330 is a module for detecting changes in viewing content output by the display apparatus 100, usage time, input source, app switching, OTT content switching, illuminance value, and the like. The illuminance sensor preprocessing module S1340 is a module for determining an illuminance range suitable for measuring biometric information using an illuminance sensor. The low-resolution movement level preprocessing module S1350 is a module for detecting whether the user is moving using a camera in a low-resolution state.

The rPPG method module S1360 is a module for measuring a biometric signal using an rPPG method from captured images of the user captured in a high-resolution state. The biometric information storage database module S1370 is a module for measuring biometric information based on a biometric signal and storing the same in a database. The measurement quality analysis module S1380 is a module for calculating a reliability score of the biometric signal using an SNR method. The viewing pattern detection module S1390 is a module for detecting a viewing pattern based on data on a user's usage state. The processor may execute such modules in parallel or sequentially to perform the above-described processes.

Meanwhile, in the various embodiments described above, a case in which biometric information is measured by a display apparatus that directly includes a camera or is connected to an external camera or an external apparatus including a camera has been described, but according to another embodiment, biometric information measurement may be performed by a server apparatus connected to the display apparatus. In this case, the display apparatus may transmit sensing results sensed by the camera and sensor to the server apparatus, or transmit data on a user state identified based on the sensing results to the server apparatus. When the data is received, the server apparatus may measure state information of the user using an artificial intelligence model, and transmit the same to the display apparatus or other user terminal apparatuses. Since specific methods of identifying a user state and measuring state information have been described in the various embodiments above, a duplicate description will be omitted.

The various embodiments described above may be implemented individually, or at least one of the embodiments may be wholly or partially combined and implemented together in one apparatus.

According to the various embodiments described above, it becomes possible to automatically determine timing suitable for biometric information measurement, thereby enabling more accurate and efficient biometric information measurement.

Meanwhile, the various embodiments described above may be applied to a product independently, or at least some of the contents may be implemented in combination with other embodiments of the present disclosure.

The above-described various embodiments may be implemented as software including instructions stored in machine-readable storage media, which can be read by machine (e.g.: computer). The machine may be a device that invokes the stored instruction from the storage medium and can be operated based on the invoked instruction, and may include an electronic device (e.g.: display apparatus (A)) according to the embodiments disclosed herein. In case that the instruction is executed by the processor, the processor may directly perform a function corresponding to the instruction or other components may perform the function corresponding to the instruction under control of the processor. The instruction may include codes generated or executed by a compiler or an interpreter. The machine-readable storage media may be provided in a non-transitory storage medium. Here, ‘non-transitory storage medium’ merely means that the storage medium is tangible without including a signal, and does not distinguish whether data are semi-permanently or temporarily stored in the storage medium.

In addition, according to an embodiment, the methods according to various embodiments described above may be included and provided in a computer program product.

Specifically, a non-transitory computer-readable storage medium or a computer program product may be provided that stores computer instructions for causing operations including displaying content according to a user input, identifying a user usage state based on a camera and at least one sensing value, identifying, based on an artificial intelligence model trained on learning data related to content viewing characteristics and the identified usage state, a state suitable for measuring biometric information, and newly obtaining image data of the user through the camera and obtaining biometric information based on the obtained image data.

The computer program product may be distributed in the form of a storage medium (e.g., compact disc read only memory (CD-ROM)) that is readable by devices, or may be distributed through an application store (e.g., PlayStore™). In the case of an online distribution, at least part of the computer program product may be at least temporarily stored in a storage medium such as a server of a manufacturer, a server of an application store, or the memory of a relay server or may be temporarily generated.

In addition, computer instructions or programs for performing the biometric information measurement method of the display apparatus according to the various embodiments described above may be stored in a non-transitory computer-readable medium. When executed by a processor of a specific apparatus, the computer instructions stored in the non-transitory computer-readable medium cause the specific apparatus to perform processing operations in the apparatus according to the various embodiments described above. The non-transitory computer-readable medium refers to a medium that stores data in a semi-permanent manner and is readable by an apparatus, rather than a medium that stores data only for a short period, such as a register, cache, or memory. Specific examples of the non-transitory computer-readable medium may include a CD, a DVD, a hard disk, a Blu-ray disc, a USB, a memory card, and a ROM.

Although preferred embodiments of the present disclosure have been shown and described above, the disclosure is not limited to the specific embodiments described above, and various modifications may be made by one of ordinary skill in the art without departing from the gist of the disclosure as claimed in the claims, and such modifications are not to be understood in isolation from the technical ideas or prospect of the disclosure.

What is claimed is:

1. A display apparatus comprising:
a display;
a camera;
at least one sensor;
a memory to store an artificial intelligence model trained based on learning data related to content viewing characteristics; and
a processor configured to:
control the display to display content according to a user input;
obtain data associated with a usage state of a user based on image data captured by the camera and sensing values of the at least one sensor; and
based on the usage state being identified as suitable to measure biometric information based on the data associated with the usage state and the artificial intelligence model, newly obtain image data of the user through the camera, and obtain the biometric information based on the newly obtained image data of the user.

2. The display apparatus of claim 1, wherein the memory stores data on a preset illuminance range; and
wherein the processor is configured to:
obtain a measurement suitability score output from the artificial intelligence model by using the data associated with the usage state as an input value of the artificial intelligence model while executing the artificial intelligence model; and
based on the measurement suitability score being equal to or greater than a preset score, and illuminance identified based on a sensing value of an illuminance sensor among the at least one sensor being included within the preset illuminance range stored in the memory, identify the usage state as being suitable to measure the biometric information.

3. The display apparatus of claim 2, wherein the processor is configured to:
based on the usage state being identified as being suitable to measure the biometric information, detect a movement of the user based on a captured image obtained through the camera in a low-resolution state; and
based on the movement of the user undetected for a preset time, obtain the biometric information of the user based on a captured image obtained through the camera in a high-resolution state.

4. The display apparatus of claim 3, wherein the processor is configured to:
identify at least one face region in a captured image obtained in the high-resolution state; and
measure the biometric information of the user from the identified at least one face region using a remote photoplethysmography (rPPG) method.

5. The display apparatus of claim 4, wherein the processor is configured to:
obtain R, G, and B pixel values of respective pixels within the identified at least one face region in consecutive multiple image frames among captured images obtained in the high-resolution state;
obtain a pulse signal according to at least one change state of the obtained R, G, and B pixel values using the rPPG method; and
obtain the biometric information of the user based on changes in magnitudes of peaks of the obtained pulse signal.

6. The display apparatus of claim 3, wherein the processor is configured to:
update the learning data using the identified usage state, and further train the artificial intelligence model using the updated learning data.

7. The display apparatus of claim 6, wherein the learning data includes:
data associated with a plurality of content viewing characteristics obtained by combining at least one characteristic among content type, a content providing source, a viewing duration, a viewing time, and an illuminance level, and a plurality of measurement suitability scores set for the plurality of content viewing characteristics, respectively; and
wherein the processor is configured to:
update the measurement suitability score according to a reliability score of the measured biometric information obtained based on signal-to-noise ratio (SNR) information calculated from the measured biometric information.

8. A method for measuring biometric information of a display apparatus, the method comprising:
displaying content according to a user input;
obtaining data associated with a usage state of a user based on a camera and at least one sensing value;
based on an artificial intelligence model trained based on learning data related to content viewing characteristics and data associated with the usage state, identifying a state suitable to measure biometric information;
newly obtaining image data of the user through the camera, and obtaining the biometric information based on the newly obtained image data.

9. The method of claim 8, comprising:
obtaining a measurement suitability score output from the artificial intelligence model by using the data associated with the usage state as an input value of the artificial intelligence model while executing the artificial intelligence model; and
based on the measurement suitability score being equal to or greater than a preset score, and illuminance identified based on a sensing value of an illuminance sensor among at least one sensor being included within a preset illuminance range stored in a memory, identifying the usage state as being suitable for measuring the biometric information.

10. The method of claim 9, comprising:
based on the usage state being identified as being suitable for measuring the biometric information, detecting movement of the user based on a captured image obtained through the camera in a low-resolution state; and
based on the movement of the user being undetected for a preset time, obtaining the biometric information of the user based on a captured image obtained through the camera in a high-resolution state.

11. The method of claim 10, comprising:
identifying at least one face region in a captured image obtained in the high-resolution state, and measuring the biometric information of the user from the identified at least one face region using a remote photoplethysmography (rPPG) method.

12. The method of claim 11, comprising:
obtaining R, G, and B pixel values of each pixel within the identified at least one face region in consecutive multiple image frames among captured images obtained in the high-resolution state;
obtaining a pulse signal according to at least one change state of the obtained R, G, and B pixel values using the rPPG method; and
obtaining the biometric information of the user based on changes in magnitudes of peaks of the obtained pulse signal.

13. The method of claim 10, comprising:
updating the learning data using data on the identified usage state, and further training the artificial intelligence model using the updated learning data.

14. The method of claim 13, wherein the learning data includes:
data associated with a plurality of content viewing characteristics obtained by combining at least one characteristic among content type, content providing source, viewing duration, viewing time, and illuminance level, and a plurality of measurement suitability scores set for the respective plurality of content viewing characteristics; and
wherein the method comprises:
updating the measurement suitability score according to a reliability score of the measured biometric information obtained based on signal-to-noise ratio (SNR) information calculated from the measured biometric information.

15. A non-transitory computer-readable recording medium storing a computer instruction executable by a processor of a display apparatus to cause the display apparatus to perform operations, the operations comprising:
displaying content according to a user input;
obtaining data associated with a usage state of a user based on a camera and at least one sensing value;
based on an artificial intelligence model trained based on learning data related to content viewing characteristics and data associated with the usage state, identifying a state suitable to measure biometric information;
newly obtaining image data of the user through the camera, and obtaining the biometric information based on the newly obtained image data.

16. The non-transitory computer-readable recording medium of claim 15, wherein the operations further comprise:
obtaining a measurement suitability score output from the artificial intelligence model by using the data associated with the usage state as an input value of the artificial intelligence model while executing the artificial intelligence model; and
based on the measurement suitability score being equal to or greater than a preset score, and illuminance identified based on a sensing value of an illuminance sensor among at least one sensor being included within a preset illuminance range stored in a memory, identifying the usage state as being suitable for measuring the biometric information.

17. The non-transitory computer-readable recording medium of claim 16, wherein the operations further comprise:
based on the usage state being identified as being suitable for measuring the biometric information, detecting a movement of the user based on a captured image obtained through the camera in a low-resolution state; and
based on the movement of the user being undetected for a preset time, obtaining the biometric information of the user based on a captured image obtained through the camera in a high-resolution state.

18. The non-transitory computer-readable recording medium of claim 17, wherein the operations further comprise:
identifying at least one face region in a captured image obtained in the high-resolution state, and measuring the biometric information of the user from the identified at least one face region using a remote photoplethysmography (rPPG) method.

19. The non-transitory computer-readable recording medium of claim 18, wherein the operations further comprise:
obtaining R, G, and B pixel values of each pixel within the identified at least one face region in consecutive multiple image frames among captured images obtained in the high-resolution state;
obtaining a pulse signal according to at least one change state of the obtained R, G, and B pixel values using the rPPG method; and
obtaining the biometric information of the user based on changes in magnitudes of peaks of the obtained pulse signal.

20. The non-transitory computer-readable recording medium of claim 17, wherein the operations further comprise:
updating the learning data using data on the identified usage state, and further training the artificial intelligence model using the updated learning data.
